(19) United States
(12) Patent Application Publication
(10) Pub. No.: US 2004/0203077 A1
(43) Pub. Date: Oct. 14, 2004

(54) Method and kit for quantitation of polypeptides

(75) Inventor: Boris Schwartsburd, Rehovot (IL)

Correspondence Address: BROWDY AND NEIMARK, P.L.L.C., 624 NINTH STREET, NW, SUITE 300, WASHINGTON, DC, 20001-5303, US

(73) Assignee: APPLIED RESEARCH SYSTEMS ARD HOLDING N. V., Curacao (NL)

(21) Appl. No.: 10/473,043

(22) PCT Filed: Mar. 25, 2002
(86) PCT No.: PCT/IL02/00246

(30) Foreign Application Priority Data
Mar. 26, 2001 (IL) 142246

(51) Int. Cl. G01N 33/53; G01N 33/567; G01N 33/569; C12N 15/87; C12N 15/74

(52) U.S. Cl. 435/7.2; 435/7.31; 435/455; 435/483

(57) ABSTRACT

The invention relates to a method for quantitation of recombinant proteins of interest in the absence of specific antibodies, which is particularly suitable for screening cell clones expressing heterologous genes.

### FIELD OF THE INVENTION

The present invention relates to a quantitative method and kit for the quantitation of recombinant polypeptides in a sample and to detection of cells expressing such recombinant polypeptides.

### BACKGROUND OF THE INVENTION

Recombinant proteins represent a versatile tool used in a variety of studies, such as those determining mechanism of action of proteins and for exploring protein-protein or protein-nucleic acid interactions. They also are often used as immunogens for the production of antibodies. In the clinic, recombinant proteins are increasingly used as therapeutic agents for the treatment of disease.

Proteins synthesised in heterologous systems can be detected either by assaying for a particular biological activity or by employing assays that are independent of such activity. In a few cases, the protein of interest carries an enzymatic or other biological activity that can be: assayed in intact cells or in extracts in vitro. Although such assays can be extremely useful, they frequently suffer from one of several practical limitations such as: (a) they may not be sufficiently sensitive to detect the small amounts of protein that are synthesized e.g. in small-scale mammalian cultures, (b) the host cells may themselves express an endogenous protein that either displays the same biological activity as the recombinant protein or interferes with it, and (c) the determination of the biological activity associated with the heterologous expression of a recombinant protein is not sufficient for establishing the exact specific activity of that protein. It is therefore essential to develop assays that are independent of biolbgical activity and sensitive enough to measure very small amounts of the protein. The reagents of choice for these assays are specific antibodies that react with the heterologous protein. Specific antibodies are commercially available for well-studied proteins. In addition, specific antibodies can be produced by immunization of appropriate animals with purified protein or with commercially available protein.

Immunologic methods for quantifying antigens provide excellent sensitivity and specificity and have become standard techniques for both research and clinical applications. All modern immunochemical methods of protein quantitation are based upon a simple and accurate method for measuring the quantity of an indicator molecule (antigen or antibody that binds to solid surfaces, such as plastics, and by washing away indicators not bound.

When the indicator molecule is labelled with a radioisotope, the assay is called a radioimmunoassay. The indicator molecule is quantified by counting radioactive decay events in a scintillation counter. The assay is called an Enzyme Linked Immunoadsorbent Assay (ELISA), when the indicator molecule is covalently coupled to an enzyme which can cleave a reporter substrate which may be calorimetric, chemiluminescent, fluorometric, or phosphometric. The indicator molecule may be quantified by determining with a spectrophotometer the initial rate at which the enzyme converts a neutral substrate to a coloured or emitting product.

ELISAs may be classified under four headings: direct, indirect, sandwich and competitive (Crowther, J. R. (1995) Methods in Molecular Biology volume 42 pages 35-50). In the direct-labelled antigen ELISA, the antibodies are adsorbed to the solid-phase and the antigen is labelled. In the direct-labelled antibody ELISA, the antigen that is attached to the solid phase is reacted directly with an enzyme labelled antibody (e.g. conjugated with an enzyme). In the indirect ELISA, the antibody is not labelled and a second antispecies specific antibody conjugated to an enzyme is used.

In the direct-sandwich ELISA, a first antibody is attached to the solid phase, the tested antigen can be added and captured by the attached antibody. A second different antibody, conjugated to an enzyme is used to detect the captured antigen. In the indirect-sandwich ELISA the second antibody is not labelled, it is generated in different animal species than the first one, and it is detected by a third antispecies specific labelled antibody.

Competitive ELISA consists of two reactants, which are competing for a third one. The following are examples of competitive ELISAs:

In the direct labelled-antibody-competitive ELISA, the antigen is adsorbed to the solid phase and a pre-titrated conjugated antibody is added, so that the antigen is saturated and no free recognition sites are available for further antibody combination. The interaction of antigen and conjugated antibody is perturbed if the labelled antibody is mixed with another antibody (competing antibody) that is able to react with the solid phase-bound antigen. Such an assay can be used to compare monoclonal antibodies directed against the same protein.

In the direct-antigen-competitive ELISA, the antigen is adsorbed to the solid phase and a pre-titrated conjugated antibody is added so that the antigen is saturated and no free antigenic sites are available for further antibody combination. In this case the interaction of antigen and conjugated antibody is perturbed if the labelled antibody is mixed in with another antigen (competitor). Thus, if the competitor antigen is cross-reactive, the labelled antibody is unavailable to react with the antigen attached to the solid phase, and a reduction in the colour is observed. Such assays are used to quantify antigens or to compare the relative affinity of binding of two antigens for the same antibody.

In the indirect-antigen/antibody-competitive ELISA, the antibody is not labelled and it is detected by a third antispecies specific labelled antibody.

The growing availability of DNA sequences and the cloning of novel genes raise the necessity of developing new tools for protein detection and purification in cases in which the information pertaining to the product of these genes is limited. The advent of tag sequences in the study of proteins has been instrumental for the efficient manipulation of recombinant proteins, notably by allowing their detection and purification in cases when no specific antibodies are available. The protein of interest can be fused to a short peptide (epitope tagged protein) or to a polypeptide (fused protein), usually with a function of its own.

The addition of specifically designed tags or the modification of sequences within the target-gene product by genetic engineering has enabled the development of novel strategies for downstream processing that can be employed for efficient recovery of both native or modified proteins.

Examples of polypeptides used to construct fusion proteins include β-galactosidase (approx. 120 kD), glutathione-s-transferase (GST approx. 26 kD Smith et al. 1988), and nmaltose binding protein (MBP approx. 44 kD Riggs P in Ausbel FM et al, eds. Current protocols in Molecular Biology 16.6.1). Fusion proteins are usually not detected by specific antibodies but rather by the function of the fused polypeptides, such enzymatic activity or by the ability to interact with another protein.

Examples of epitope tags are histidine tags (a stretch of 6 consecutive histidines. Janlknecht et al. 1991), FLAG (S aminoacid epitope ROCHE). VSV-G (11 amino acid epitope from vesicular stomatitis virus ROCHE), Protein-C (12 aminoacid epitope from the heavv chain of human Protein C., ROCHE), and c-myc (10 aminoacid epitope from the human c-myc gene protein ROCHE). In an epitope tagged protein the added sequence is a short peptide of about 3-12 amino acids, usually with no function of its own. The important property of the epitope tag is its ability to be recognized and bound by a single, tag-specific antibody. Epitope tags can be placed at the amino-terminus (N-terminus), at the carboxy-terminus (C-terminus), or within the coding region of the protein.

A wide range of epitopes has been used to tag proteins and many tag specific antibodies are commercially available. The best epitope tag for a particular experimental system is the one that does not interfere with the function or cellular processing of the tagged protein, yet generates a strong detection signal on Western blots, in immunofluorescence microscopy, or in other analytical techniques.

Epitope tags are incorporated at the DNA level. To fuse the coding sequence of the epitope to that of the protein of interest, simple recombinant DNA techniques or oligonucleotide-mediated mutagenesis via Polymerase Chain Reaction (PCR) are used.

The DNA sequence encoding the tagged protein is cloned into an expression vector comprising appropriate transcriptional and translational regulatory sequences. The expression vectors are introduced into a suitable host (prokaryotic or eukaryotic host).

The common assays that use epitope tagging, are western blotting, immunofluorescence microscopy, immunoprecipitation (Sells and Cemoff, 1955, Chubet and Brizzard, 1996), and affinity chromatography.

Biological applications for epitope tagging methodology comprise: determining the size, intracellular localization, abundance of proteins (Molloy et al 1994, Canfield et al. 1996), monitoring post-translational modification, analyzing the function of individual protein domains, studying receptor binding and internalization of exogenous proteins (Brown et al. 1995), and establishing the identity of a protein within a protein complex (Zhou et al 1992).

One of the most widely used molecular tagging methods, is the expression or recombinant proteins as fusion molecules with histidine tags which typically consists of five or six consecutive histidine residues (Hoclhuli 1958). The resultant proteins, containing the histidine residues have useful properties such as the ability to chelate the free coordination sites of metal ions which are themselves immobilized as chelate complexes of iminodiacetic acid (IDA) or nitriloacetic acid (NTA) bound to solid support (reviewed in Arnold 1991). Their ability is conferred by the imidazole moieties of the histidines. Typically Ni⁺², Zn⁺², Co⁺² and Cu⁺² chelated to IDA or NTA have been used as a chromatographic matrix for affinity purification of the protein under study.

In addition to the exploitation of the affinity of histidine moieties to metals, antibodies are now available recognising the histidine tags (Lindner et al. 1997). Such polyclonal and monoclonal antibodies are employed for the detection of histidine tagged proteins immobilised on western blots.

Methods are known for quantitation of histidine-tagged proteins. For example, a sample containing the poly histidine tagged protein is loaded into a metal coated surface (Reacti-Bind™ Metal Chelate Plates, Pierce). After washing away the non bound material the histidin-tagged protein can be detected by an antibody specific to the histidine tag or alternatively by the INDIA™ HisProbe-HRP which is a nickel 2+-activated derivative of horseradish peroxidase. This approach lacks specificity and allows non-specific binding of proteins, which contain histidines in their natural sequence to the nickel-coated surface.

The use of tags for the quantitation of recombinant bacterially expressed proteins is known from the QIAexpress Assay System (QIAGEN), which exploits the strong interaction between 6× His tag and Ni-NTA to immobilize functionally active proteins and other biomolecules for improved assays. 6xhistidine-taaged biomolecules can be selectively captured from complex mixtures of molecules, such as cell lysates. The biomolecules are bound in a uniform orientation offering optimal presentation of binding domains to interacting partners, resulting in higher signal-to-noise ratios and reliable, reproducible results. Proteins that are expressed from the pQE-100 DoubleTag Vector have an N-terminal 6× His tag and a C-terminal Tag 100. These doubly tagged proteins are conveniently immobilized on Ni-NTA HisSorb Strips and Plates or Ni-iNTA Magnetic Agarose Beads via the 6×His tag and detected using the Tag 100 Antibody. This method eliminates the need for antibodies specific to the protein of interest, but still has the limitation of lacking specificity by allowing non-specific binding of proteins that contain histidine stretches in their natural sequence to the nickel coated surface. Furthermore, the presence of two tags may affect the conformation or the biololgical activity of a given protein.

Currently no general, sensitive, specific, quantitative easy to perform and efficient method exists which allows detection of cells producing recombinant polypeptide in the absence of specific antibodies.

Therefore the method described in the present invention solves a long-standing problem in the area of recombinant protein quantitation when specific antibodies are not available, as e.g. in the case of proteins encoded by novel genes.

### SUMMARY OF THE INVENTION

The invention relates to a method for determining the amount of an histidine-tagged polypeptide in cells, body fluids or in a fraction resulting from downstream processes comprising.

a) expressing a recombinant histidine-tagged polypeptide in a cell,

b) incubating a mixture of a sample containing the recombinant histidine-tagged polypeptide with an anti His-tag specific antibody, appling the mixture to a solid phase surface coated with monomeric or polymeric histidine tag or carrier proteins containing histidine tag,

c) separating said mixture from said solid phase,

d) detecting and measuring the amount of anti His-tag specific antibody bound to said solid phase, and

e) determining the amount of histidine-tagged polypeptide in said sample from the amount of antibody bound to said solid phase.

The polypeptide the amount of which is to be determined may be encoded by a cDNA, genonic DNA, EST or a DNA with an open reading frame and the sample tested is selected from a cell lysate and conditium medium.

In one embodiment the polypeptide is fused to a histidine tag comprising six consecutive histidines at the C-terminal or N-terminal domain and expressed in eukaryotic and prokaryotic cells. In a preferred embodiment the polypeptide is fused to a histidine-tag and is produced in eukaryotic cells selected from yeast, fungi, plant, BHK, COS, HeLa and CHO cells.

In one aspect of the invention, the antibody, polyclonal, monoclonal, Fab fragment or a any other antibody fragment may be labelled itself and alternatively, a labelled secondary-antibody may be used to detect the first unlabelled antibody or fragment thereof. In one

### BRIEF DESCRIPTION OF THE FIGURES

FIG. 1. shows a schematic representation of the competitive ELISA according to the invention.

FIG. 2. shows the titration of anti histidine (6× His) specific Mab and coating antigen (poly-histidine) to determine the optimal concentrations of these reagents to be used in competitive ELISA. The binding of anti histidine specific Mab (expressed as OD) to poly-histidine coated plates, as a function of antibody concentration, was tested at different concentration of poly-histidine used to coat the microtiter plates (0.125, 0.25, 0.5 and 1 μg/ml).

FIG. 3A shows the dose-response curve for IL-18BP-His inhibition ELISA. Dose response curve was obtained as follows: 100 μl of serial diluted competitor, IL-18BP-His (from 0.19 μg/ml to 100 μg/ml) and 100 μl of Mab (62.5 ng/ml) were mixed and incubated in poly-histidine coated plates. Following incubation, the plates were washed and the antibody bound to the plates was detected using HRP-conjugated AffinitiPure Goat anti mouse IgG and its specific substrate. The graph represents logit-log plot of absorbency (O.D. at 492 nm) as a function of concentration of competing IL-18BP-His (μg/ml). The O.D. was calculated as the average of duplicate measurements.

FIG. 3B shows the dose-response curve for BID-His inhibition ELISA. The dose response curve was obtained as described in FIG. 3A using BID-His as the competitor.

FIG. 3C shows the dose-response curve for Sp Lyt A-His inhibition ELISA. The dose response curve was obtained as described in FIG. 3A using Sp Lyt A-His as the competitor.

FIG. 3D shows the dose-response curve for Mur D-His inhibition ELISA. The dose response curve was obtained as described in FIG. 3A using Mur D-His as the competitor.

### DETAILED DESCRIPTION OF THE INVENTION

The invention relates to a quantitative method for the detection of recombinant proteins in a sample and for the detection of cells producing recombinant proteins. This method is suitable for detecting cell clones expressing heterologous genes encoding novel proteins and/or polypeptides in both prokaryotic and eukaryotic systems.

The first step of the method consists of the fusion of a cDNA sequence, genomic DNA, or a novel gene or EST with open reading frames encoding a polypeptide to a DNA sequence encoding all histidine tag, which typically consists of four or more consecutive histidine residues. The tag can be fused to the N-terminal or C-terminal end of the encoded polypeptide of interest. If desired, sequences recognized by a site specific proteolitic enzyme such as Xa (WO 00/61768) can be inserted between the tag to allow in vitro Xa cleavage and recovery of untagged protein. Alternatively, the proteolitic enzyme used can be caspase-8, a cysteine aspartate-specific proteinases (Nicholson DW et al. 1997, Salvesen GS et al. 1997, Cohen GM 1997). The cleavage sites of caspases are defined by tetrapeptide sequences (X-X-X-D) and cleavage always occurs downstream of the aspartic acid.

Alternatively, the tag can be placed within the coding region of the gene, provided that it does not effect the activity of the protein. Alternatively, an endogenous-natural stretch of histidine residues can be used as a tag as well.

Expression vectors designed for the production of histidine tag fusion proteins are commercially available. These vectors encode a histidine tag up-stream or down-stream of the cloning site. Alternatively, the histidine tag may be fused to the gene of interest by polymerase chain reaction (PCR), for example by including the histidine tag sequence in the primers used for the amplification of the DNA of interest. The PCR amplified DNA fusion product may be cloned into an expression vector comprising appropriate regulatory signals for transcription and translation. The tagged protein/s of interest can be expressed in suitable host cells which have been transfected, transformed or infected with the appropriate expression vector carrying the DNA sequence of the histidine tagged protein/s of interest and cultured. The recombinant proteins produced can be soluble, insoluble (inclusion bodies), intracellular, periplasmic or secreted to the medium.

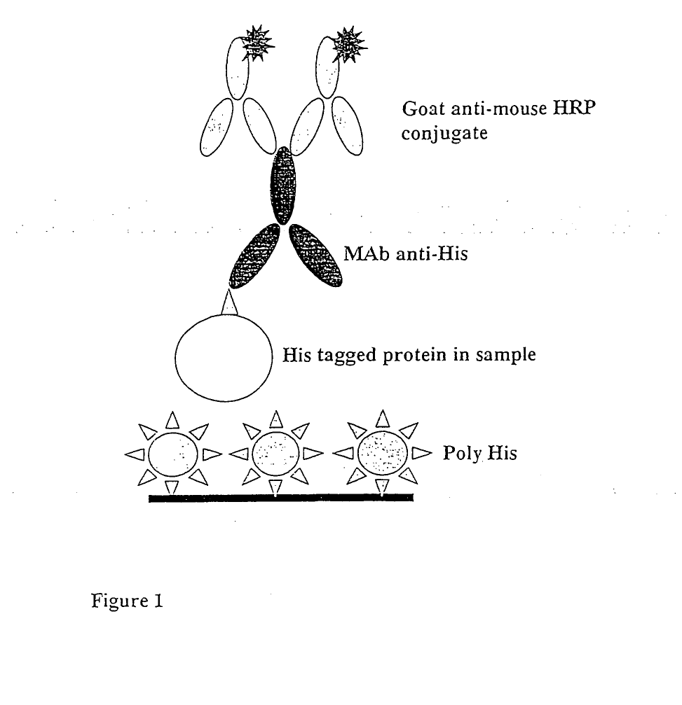

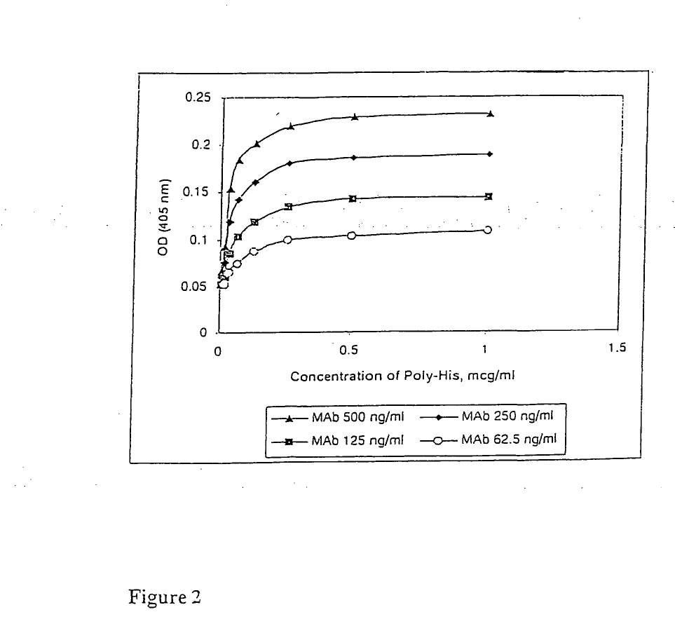

The second step of the method consists of the quantitation of the recombinant protein/s by a competitive immunoassay (see schematic representation in FIG. 1). In this assay the histidine-tagged protein in the sample, usually the supernatant of transfected cells competes for the binding of anti histidine specific antibodies to a histidine coated solid phase surface, e.g. 96-well microtiter plates.

For better quantitation of the histidine tagged polypeptides produced by the transfectd cells, the optimal concentration of the reagents to be used in the competitive immunoassay e.g. that of anti His-tag specific antibody and the histidine used to coat the plates, is determined. The optimal antibody and histidine concentrations desired are those in which the antibodies are saturated by the histidine bound to the solid surface and no free antibody is available for further combination with antigen.

The amount of histidine-tagged protein in a sample is measured by pre-incubating or mixing anti His-tag specific antibodies (at optimal concentration as described above) with the sample. The mixture of the antibody and the tested sample is loaded and incubated on

The anti His-tag specific antibodies can be polyclonal or monoclonal, can be represented by an antibody fragment such the Fab fragment, they may be commercial or in house prepared.

The tested samples may be crude preparations, such as supernatant of cell cultures cell lysates, body fluids (e.g. injected histidine-tagged protein in animals or individuals) or may be fractions resulting from downstream purification processes.

Labelling may be carried out by using enzymes linked to antibody or protein A/G and visualized by coloured, florescent or chemiluminescent specific substrates. Alternatively, the antibody or protein A/G may be radioactively labelled. In addition, biotin may be chemically linked to the antibody or protein A/G trough lysine residues. Labelled streptavidine may be used to detect the presence of biotin.

The method according to the invention may facilitate high throughput screening of recombinant target and narrow the number of tested samples by screening of target proteins only in these in cells that after transfection with an expression library express recombinant

The hisitine-tagged proteins itself may be used to explore the therapeutic value of the novel protein and its mechanism of action.

The invention also provides a kit for the quantitation of recombinant proteins comprising the following components: 1) precoated 96-well plates with optimal amount of poly-histidine 2) anti-histidine antibody 3) instructions for titration of optimal antibody and poly-histidine concentrations 4) a purified histidine tagged protein reference 5) instructions for the dose response curve generation. The kit may optionally contain washing buffer, HRP-conjugate affinity pure Goat anti mouse IgG and substrate solution, nickel columns for affinity purification and a suitable vector for generating the histidine tag fusion.

The invention is illustrated by the following non-limiting examples.

TABLE I

| | | | | Localisation of | |
|---|---|---|---|---|---|
| | | | MW | His × 6 fusion | Expression |
| No. | Protein | Function | (kD) | Tag | system |
| 1 | IL-1SBP | IL-18 binding protein. | 28.0 | C | CHO |
| 2 | BID | BH3 domain of Bcl-2 associated plasma | 22.8 | N | E. coli |
| | | membrane protein with pro-apoptotic | | | |
| | | activity. | | | |
| 3 | Sp Lyt A | (N-acetylmuramoyl-1-alanine amidase) which is | 370 | C | E. coli |
| | | an autolysin of Streptococcus pnewnoniae. | | | |
| 4 | Mur D | (UDP-N-acetylmuramoyl-1-alanine:d-glutamate) | 54.6 | C | E. coli |
| | | which is a ligase catalysing the addition of | | | |
| | | D-glutamate to the nucleotide precursor UDP-N- | | | |
| | | acetylmuramoyl-1-alanine (UMA). | | | |

with different concentrations of poly-histidine, and incubated for 1 hour at 37° C., with shaking. The plates were then washed four times with 300 μl/well washing buffer and incubated with 100 μl/well of HRP-conjugated AffiniPure Goal anti mouse IgG (HRP) diluted 1:10,000 in assay buffer for 1 hour at 37° C., with shaking. After removing the excess of the antibody by washing, the detection-substrate solution was added until color developed. The reaction was stopped by adding 125 μl/well of 4N HCl. Absorbency was measured at 492 nm in an ELISA reader with a reference wavelength of 405 nm. A curve of OD as a function of poly-listidine concentration used to coat the plates was generated for each concentration of anti His-tag specific antibody (FIG. 1). The titration shows a plateau region at high poly-histidine concentration, where increasing concentration of poly-histidine has no effect on the scored values. The plateau represents the region where the entire antibody is saturated with poly-histidine; which indicates that the antibody is limiting. The dilution of antibody selected for the competitive ELISA was 62.5 ng/ml and the concentration of poly-histidine used to coat the plates was 5 μpg/ml. histidine) and incubated for 1 hour at 37° C., with shaking. After this incubation period, the plates were washed four times with 300 μl/well washing buffer and incubated with 100 μl/well of HRP-conjugated AffiniPure Goat anti mouse IgG (HRP diluted 1:10,000 in assay buffer) for 1 hour at 37° C., with shaking. After washing the excess of the antibody, the substrate for HRP was added until colour developed. The reaction was stopped by adding 125 μl/well of 4N HCl. Absorbance (OD) was measured at 492 nm in an ELISA reader with a reference wavelength of 405 nm. The absorbance of the negative controls (all reagents except the antibody) provided the background values. The average O.D was calculated from duplicate measurements.

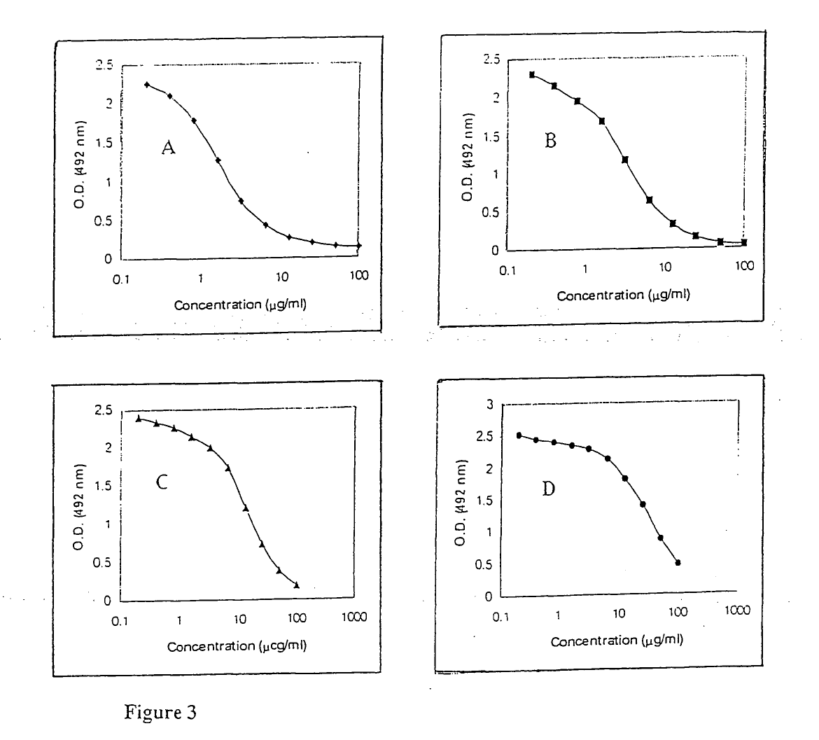

The graphs shown in FIGS. 3A, 3B, 3C and 3D represent logit-log plots of absorbance as a function of the concentration of the four different recombinant proteins (competitors). The curves show sigmoidal appearance. It can be seen that the OD obtained is inversely proportional to the concentration of competitor (histidine-tagged proteins). 100% competition crude preparations. An example of a crude preparation is the condition media of IL-1SBP secreting cell cultures.

TABLE 2

| Concentration of | Measured OD (492 nm) | Measured OD (492 nm) in |
|---|---|---|
| IL-18E (μg/ml) | buffer | 10% serum |
| 0.00 | 2.283 ± 0.005 | 2.103 ± 0.035 |
| 0.75 | 0.913 ± 0.008 | 0.998 ± 0.013 |
| 1.50 | 0.639 ± 0.025 | 0.651 ± 0.009 |
| 3.00 | 0.411 ± 0.008 | 0.427 ± 0.008 |

Following gene amplification, the best producer cells are selected and tested for stability of production after MTX withdrawal using the competitive imunoassay. Cells expressing stable levels of recombinant protein upon propagation in the absence of MTX are selected for cloning. The best clone is selected for production of the recombinant polypeptide.

### REFERENCES

Arnold. F. H. (1991). “Metal-affinity separations: a new dimension in protein processing.” Biotechnology (NY), 9(2), 151-6.

Botting, C. H., and Randall, R. E. (1995). “Reporter enzyme-nitrilotriacetic acid-nickel conjugates: reagents for detecting histidine-tagged proteins.” Biotechniques, 19(3), 362-3.

Goel,a., Beresford, G. W., Colchler, D., Pavlinkova, G., Booth, B. J. M., Baranowska-Kortylewicz, J. and K. B., Surinder (2000). “Divalent Forms of CC49 Single-Chain Antibody Constructs in Pichia pastoris: Expression, Purification, and Characterizationl” J. Biochem. 127, 829-36.

Hochuli, E. (1988). “Large-scale chromatography of recombinant proteins.” J Chromatogr, 444,293-302.

Janknecht, R., de Martynoff, G., Lou, J., Hipskind, R. A., Nordheim, A., and Stunnenberg, H. G. (1991). “Rapid and efficient purification of native histidine-tagged protein expressed by recombinant vaccinia virus.” Proc Natl Acad Sci USA, 88(20), 8972-6.

Lindner, P., Bauer, K., Krebber, A., Nieba, L., Kremmer, E., Krebber, C., Honegger, A., Klinger, B., Mocilkat, R., and Pluckthun, A. (1997). “Specific detection of his-tagged proteins with recombinant anti-His tag scFv-phosphatase or scFv-phage fusions.” Biotechniques, 22(1), 140-9.

Nygren, P. A., Stahl, S., and Uhlen, M. (1994). “Engineering proteins to facilitate bioprocessing.” Trends Biotechnol, 12(5), 184-8.

Smith, D. B., and Johnson, K. S. (1988). “Single-step purification of polypeptides expiessed in Escherichia coli as fusions with glutathione S-transferase.” Gene, 67(1), 31-40.

1. A method for determining the amount of an histidine tagged polypeptide in cells body fluids or in a fraction resulting from downstream processes comprising:
a) expressing a recombinant histidine-tagged polypeptide in a cell,
b) incubating a mixture of a sample containing the recombinant histidine-tagged polypeptide with an anti His-tag specific antibody, applying the mixture to a solid phase surface coated with monomeric or polymeric histidine tag or carrier proteins containing histidine tag,
c) separating said mixture from said solid phase,
d) detecting and measuring the amount of anti His-tag specific antibody bound to said solid phase, and
e) determining the amount of histidine-tagged polypeptide in said sample from the amount of antibody bound to said solid phase.

2. A method according to claim 1, wherein the polypeptide is encoded by a cDNA, genomic DNA, EST or a DNA with an open reading frame.

3. A method according to claim 1 or 2, wherein the sample is selected from a cell lysate and condition medium.

4. A method according to anyone of claims 1 to 3, wherein the polypeptide is produced in a eukaryotic cell.

5. A method according to claim 4, wherein the polypeptide is produced in yeast or fungi.

6. A method according to claim 4, wherein the poypeptide is produced in plant cells.

7. A method according to claim 4, wherein the polypeptide is produced a mammalian cell.

8. A method according to claim 7, wherein the mammalian cell is selected from CHO, BHK, COS and HeLa cells.

9. A method according to anyone of claims 1 to 3, wherein the polypeptide is produced in a prokaryotic cell.

10. A method according to anyone of claims 1 to 9, wherein the histidine-tag comprises 6 consecutive histidines.

11. A method according to claim 10, wherein the antibody is specific to a tag comprising six consecutive histidines.

12. A method according to anyone of claims 1 and 11, wherein the antibody is polyclonal, monoclonal, Fab fragment or other fragment thereof.

13. A method according to anyone of claims Hand 12, wherein the antibody is labelled.

14. A method according to claim 12, wherein the antibody is detected by a labelled secondary-antibody.

15. A method according to claim 14, wherein the secondary-antibody is an anti species IgG specific antibody.

16. A method according to anyone of claims 13 to 15, wherein the label is selected from the group consisting of radioisotopes, catalysts, fluorescent compounds, chemiluminiscent compounds, enzymes, and enzyme substrates.

17. A method according to claim 16, wherein detection of antibody bound to said solid phase is carried out by reacting with a HRP-conjugate AffiniPure Goat anti mouse IgG (HRP) and substrate solution (OPD).

18. A method according to anyone of claims 1 to 17, wherein the solid phase is coated with Poly-L-histidine.

19. A method according to claim 18, wherein the solid phase is a microtiter plate.

20. A method according to anyone of claims 1 to 19, wherein the polypeptide is histidine-tagged at the C-terminal domain.

21. A method according to anyone of claims 1 to 19, wherein the polypeptide is histidine-tagged at the N-terminal domain.

22. A method according to anyone of claims 1 to 21, wherein the polypeptide is IL-18BP.

23. A method according to anyone of claims 1 to 22, wherein a recognition site specific for a proteolitic enzyme is inserted in the histidine-tagged protein between the histidine-tag and the polypeptide, allowing recovery of the untagged protein.

24. A method according to claim 23, wherein the proteolitic enzyme is Xa.

25. A method according to claim 23, wherein the proteolitic enzyme is caspase-S.

26. A kit for performing a histidine-tagged polypeptide competitive immunoassay comprising:
a) a solid phase coupled to monomeric or polymeric histidine-tag or carriers containing histidine-tag,
b) an anti His-tag specific antibody
c) positive reference histidine-tagged polypeptide, and
d) instructions.

27. A kit according to claim 26, wherein the reference polypeptides are selected from, IL-18BP-His, BID-His, Sp Lyt A-His or MurD-His.
